(19) United States
(12) Patent Application Publication
(10) Pub. No.: US 2006/0292189 A1
(43) Pub. Date: Dec. 28, 2006

(54) Ophthalmic solution with a flavoring agent as a dosing indicator and method for indicating dosage of an ophthalmic solution

(75) Inventors: Erning Xia, Penfield, NY (US); Joseph C. Salamone, Boca Raton, FL (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/431,950

(22) Filed: May 11, 2006

Related U.S. Application Data
(60) Provisional application No. 60/687,042, filed on Jun. 3, 2005.
(60) Provisional application No. 60/704,846, filed on Aug. 2, 2005.

(51) Int. Cl. A61K 9/00

(52) U.S. Cl. 424/400

(57) ABSTRACT

The invention provides an ophthalmic solution with a flavoring agent as a dosing indicator and a method for indicating dosage of an ophthalmic solution. To indicate dosage, a flavoring agent is added to an ophthalmic solution in an amount correlated to the dosage of the ophthalmic solution to be used. The flavoring agent can be a sweet flavoring agent (sweetener), or combinations of a sweetener with a sour flavoring agent or a bitter flavoring agent or mixtures thereof. The flavoring agent may also be natural flavors, natural fruit flavors, artificial flavors, artificial fruit flavors, flavor enhancers and mixtures thereof, which may be combined with a sweetener, a sour flavoring agent, a bitter flavoring agent, and mixtures thereof. The invention may be utilized as a contact lens solution, an eye drop formulation, and a pharmaceutical composition containing at least one active pharmaceutical ingredient for the treatment of eye diseases. The invention also provides to a method of administering a dose of an ophthalmic solution to the person's eye.

### CROSS REFERENCE

This application claims the benefit of Provisional Patent Application No. 60/687,042 filed Jun. 3, 2005 and Provisional Patent Application No. 60/704,846 filed Aug. 2, 2005 and are incorporated herein by reference.

### FIELD OF INVENTION

The invention related to an ophthalmic solution with a flavoring agent as a dosing indicator and a method for indicating dosage of an ophthalmic solution. The invention also relates to a method of administering a dose of an ophthalmic solution to a person's eye.

### BACKGROUND

Ophthalmic solutions include a wide variety of aqueous formulations for an eye and a contact lens care as well as many therapeutic treatments. For example, ophthalmic solutions can be formulated as a pharmaceutical composition containing at least one active pharmaceutical ingredient for the treatment of an eye disease. Isotonic solutions for improving the comfort of wearing soft contact lenses by being added directly to the contact lens in the eye are well known. Various ophthalmic solutions including a contact lens solution and an eye drop formulation have been developed over the years to ensure that contact lenses are essentially pathogen and deposit free. So-called, multipurpose solutions (MPS) can disinfect and clean without harming the eye or lens in addition to wetting.

These contact lens solutions commonly include anti-microbial substances as well as cleaning (active against both lipids and proteins), wetting and other agents for the disinfection and cleaning of contact lenses during storage after wear. The solution must be “ophthalmically safe” for use in the eye or with a contact lens, meaning that a contact lens treated with the solution is generally suitable and safe for direct placement on the eye without rinsing. As ophthalmic solutions typically contain viscosity enhancing agents, lubricants, surfactants, buffers, preservatives, and salts as inactive ingredients, ophthalmic solution may have its own flavor or not have any flavor.

Ophthalmic solutions are generally administered by means of a plastic bottle with an attached dropper. The maximum volume of a solution that can be added into the lower eyelid sack is generally 30 μl, although it depends on various factors such as the structures and conditions of a person's eyes. An excess of the solution administered is eliminated via nasal drainage, which eventually flows to the mouth. Administration of an eye drop is always difficult because there is no dosage indicator in conventional ophthalmic solutions. It is therefore desirable to provide the palatable ophthalmic solutions with a variety of flavors act as a dosing indicator.

### SUMMARY OF INVENTION

The invention relates to an ophthalmic solution with a flavoring agent as a dosing indicator and a method for indicating dosage of an ophthalmic solution. To indicate dosage, a flavoring agent is added to an ophthalmic solution in an amount correlated to the dosage of the ophthalmic solution to be used. The invention also includes a method of administering an ophthalmic solution to a person's eye until a taste sensation occurs in the patient's mouth.

### BRIEF DESCRIPTION OF THE DRAWINGS

FIG. 1 illustrates the portions of the human mouth and throat which respond to different tastes.

### DETAILED DESCRIPTION OF THE INVENTION

The invention provides a method for indicating dosage of an ophthalmic solution, comprising the step of adding a flavoring agent to an ophthalmic solution in an amount correlated to the dosage of the ophthalmic solution to be used. A flavoring agent in an ophthalmic solution in an amount to generate a taste sensation when the proper dosage of the ophthalmic solution is administered to a patient. The flavoring agent may incorporated into an ophthalmic solution by simply adding an appropriate amount of the flavoring agent to an existing ophthalmic solution, e.g. when dispensed at a pharmacy. Alternatively, the flavoring agent may be added when the ophthalmic solution is originally manufactured. The amount of flavoring, then, correlates to the dosage of the ophthalmic solution to be administered to a person. The method of the invention further provides the incorporating step comprises adding a flavoring agent to a prepared ophthalmic solution.

According to the invention, the flavoring agent may be formulated into a contact lens solution, an eye drop formulation, and a pharmaceutical composition containing at least one active pharmaceutical ingredient for the treatment of an eye disease. The flavoring agent may be added to an already prepared ophthalmic solution or be added during the preparation of the ophthalmic solution using formulation techniques known in the art. In general, this requires only simple mixing. In a pharmaceutical composition for the treatment of an eye disease, the amount of at least one active pharmaceutical ingredient correlates to the dosage of the ophthalmic solution to be administered to a patient. The flavoring agent is suitably present in an amount to cause taste, for example, from about 0.0001 to 20 weight percent. Preferably, the flavoring agent may be present in an amount ranging from 0.01 to 10 weight percent, more preferably in an amount from 0.5 to 5 weight percent, and most preferably in an amount from 0.1 to 5 weight percent. U.S. Pat. Nos. 5,604,189, 4,820,352, and 6,037,328 disclose the representative contact lens solutions. Typical eye drop formulations are disclosed in U.S. Pat. Nos. 6,348,508, and 3,987,163. Pharmaceutical compositions for the treatment of an eye disease are disclosed in U.S. Pat. Nos. 4,960,799, 6,872,383 and 6,861,411.

Flavor is the sensation caused by those properties of any substance taken into the mouth which stimulates one or both of the senses of taste and smell and/or also the general pain, tactile, and temperature receptors in the mouth. There are four major tastes; (1) sourness, (2) sweetness, (3) saltiness, and (4) bitterness. Sourness is the simplest taste. Usually, the more hydrogen ions, the sourer the solution becomes. The most common taste activators for sweetness are sugars, although there are many other compounds that have sweet taste. Sodium chloride has the most pure salty taste. The examples of the compounds for bitterness are caffeine, nicotine, quinine and brucine.

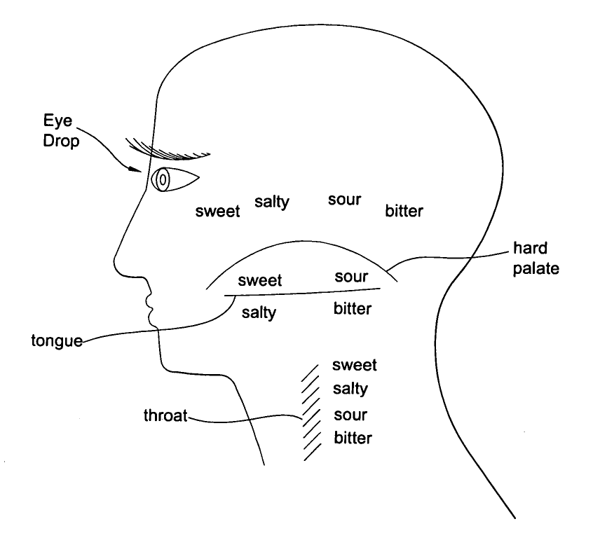

FIG. 1 depicts the portions of the human mouth and throat which respond to different tastes. Taste, or gustation, is the combined impression we receive when free nerve endings and taste buds in the mouth detect various stimuli. The free nerve endings possess no receptors, but are responsible for the perception of sensations such as pain, temperature, pungency, and astringency. The taste buds are clusters of approximately 100 taste cells that occur as protuberances, called papillae, on the tongue. Taste cells lie within taste buds, which are located in various tongue papillae, hard and soft palate, and root of the tongue. The mechanism of flavor perception is not well understood, but it is believed that the arrival of a chemical stimulant on the surface of a receptor temporarily modifies the cell wall and produces an electrochemical impulse. This impulse is then transmitted through a nerve cell to the brain, where it is decoded into sensory information in the cerebral cortex. Taste depends mainly on the contact of soluble matter with the terminal organs (connected with branches of the glossopharyngeal and other nerves) in the papillae on the surface of the tongue. The four basic tastes (sweet, salt, sour, and bitter) are unevenly distributed on the tongue, as seen in FIG. 1. The base of the tongue is considered most sensitive to bitter substances, the point to sweet and acid substances.

Flavoring agents may be a single chemical or a blend of chemicals whose primary purpose is to provide all or part of the particular flavor or effect to any products such as ophthalmic products. The flavoring agent can be in the forms of oils or extracts. The flavoring agent may be acidic, basic, neutral or salt.

The flavoring agent may be selected from four major tastes: sourness, sweetness, saltiness, and bitterness, or combination thereof. Preferably, the flavoring agent has sweetness by a sweet flavoring agent (a sweetener), a combination of a sweetener with other flavoring agents, or mixtures thereof. Most preferably, the flavoring agent may be a sweetener, a combination of a sweetener with a sour flavoring agent, a combination of a sweetener and a bitter flavoring agent, or mixtures thereof. In addition, the flavoring agents can be selected from the group consisting of natural flavors, natural fruit flavors, artificial flavors, artificial fruit flavors, flavor enhancers and mixtures thereof.

The flavoring agent may be a sweetener which may be preferably used to mask the inherent flavors of the ophthalmic solution. Sweeteners are the food additives of natural sugar, or sugar substitutes of artificial origin. The invention provides an ophthalmic solution with a flavoring agent, wherein the flavoring agent is a sweetener.

The sweetener used may be selected from a wide range of materials including water-soluble sweeteners, water-soluble artificial sweeteners, water-soluble sweeteners derived from naturally occurring water-soluble sweeteners, and mixtures thereof. Without being limited to particular sweeteners, representative categories and examples are shown in Table 1.

TABLE 1

| Water-soluble Sweeteners | | Water-soluble Sweeteners |
|---|---|---|
| (monosaccharides, | | derived from naturally |
| disaccharides and | Water-soluble | occurring Water-soluble |
| polysaccharides) | Artificial Sweeteners | Sweeteners |
| xylose, ribulose, glucose | soluble saccharin salts, i.e., | chlorinated derivatives of |
| (dextrose), mannose, | sodium or calcium saccharin | ordinary sugar (sucrose), |
| galactose, fructose (levulose), | salts, cyclamate salts, the | known, for example under the |
| sucrose (table sugar), maltose, | sodium, ammonium or | produce designation of |
| invert sugar (a mixture of | calcium salt of 3,4-dihydro-6- | sucralose. |
| fructose and glucose derived | methyl-1,2,3-oxathiazine-4- | |
| from sucrose), partially | one-2,2-dioxide, the | |
| hydrolyzed starch, corn syrup | potassium salt of 3,4-dihydro- | |
| solids, dihydrochalcones, | 6-methyl-1,2,3-oxathiazine-4- | |
| monellin, steviosides, | one2,2-dioxide (Acesulfame- | |
| glycyrrhizin, and sugar alcohols | K), the form of sacchrin, and | |
| such as sorbitol, mannitol, | the like. | |
| maltitol, hydroganated starch | | |
| hydrolysates and mixtures | | |
| thereof. | | |

Preferred sugar based sweeteners in the invention are dextrose, sucrose, and fructose and mixtures thereof. Most preferably, the sweetener is sucrose.

The use of artificial sugar products as the flavoring agent provides an ophthalmic solution for persons concerned with diet management. Artificial/synthetic sweeteners, sugar alternatives, alternative sweeteners, non-nutritive sweeteners, non-caloric/low-cal/low-carb sweeteners, diabetic-safe sweeteners are all interchangeable and synonymous for the purposes of the invention. There are currently five low-calorie sweeteners approved by the Food and Drug Administration (FDA), including acesulfame potassium, aspartame, neotame, saccharin and sucralose. These sweeteners are hundreds of times sweeter than sucrose and do not contribute calories to the diet. Sucralose, chemically known as 1,6-dichloro-1,6-dideoxy-BETA-D-fructofuranosyl-4-chloro-4-deoxy-alpha-D-galactopyranoside, is a non-nutritive, high-intensity sweetener made from a process that begins with sucrose and sold under the Splenda® trademark. The chemical structures of sugar and sucralose are: 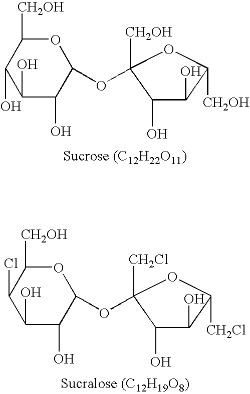
Sucralose contains tightly bound chlorine atoms, which create a sweetener that is 600 times sweet than sugar.

There are also a number of reduced-calorie sweeteners (polyols) available in the U.S., including erythritol, hydrogenated starch hydrosylates, isomalt, lactitol, maltitol, mannitol, sorbitol and xylitol. Polyols contribute between and 0.2 and three calories per gram as opposed to sucrose, which contributes four calories per gram. Polyols not only contribute sweetness but also bulk, and are used in a variety of products.

Sugarless sweeteners in the invention may include, but are not limited to, are sucralose, isomalt, aspartame, saccharin, lacitol, high-fructose corn syrup and other sweet replacers.

As discussed above, any flavoring agent or combination of flavoring agent may be used in the ophthalmic solution of the invention. Without being limited to particular flavors, examples of flavoring agents are shown in Table 2 (available from International Flavors & Fragrances, Inc.

(http://www.iff.com/_{—}85256C33004F6FEB.NSF/FlavIngredients!OpenForm).

TABLE 2

| | |
|---|---|
| 6-Methyl Coumarin | Artificial & Kosher |
| Anethole USP | |
| Cassia Oil | Natural & Kosher |
| Cassia Oil Redistilled | Natural & Kosher |
| Cinnamon Bark Oil | Natural & Kosher |
| Clove Bud Oil English Distilled SAS | Natural & Kosher |
| Clove Leaf Oil Redistilled | Natural & Kosher |
| Cocoa Distillate (Nat.) | Natural & Kosher |
| Cocoa Essence Dark | Natural & Kosher |
| Cocoa Essence White | Natural & Kosher |
| Coriander Oil | Natural & Kosher |
| delta Decalactone | Natural & Kosher Parve |
| Dimethyl Benzyl Carbinyl Butyrate | Artificial & Kosher Parve |
| Ethyl-2-Methyl Butyrate | Natural & Kosher Parve |
| Ethyl-3-Hydroxy Butyrate | Artificial & Kosher Parve |
| Ethyl Butyrate | Natural & Kosher Parve |
| Ethyl Iso Butyrate | Natural & Kosher Parve |
| Ethyl Iso Valerate | Natural & Kosher Parve |
| Ethyl Oxanoate 369 | Artificial & Kosher Parve |
| Eucalyptus Oil 80% | Natural & Kosher |
| Farnesene 1% PG/ETOH | Artificial & Kosher Parve |
| Furfurrole 302 | Artificial & Kosher Parve |
| gamma-Decalactone | Natural & Kosher Parve |
| gamma-Hexalactone | Natural & Kosher Parve |
| gamma-Octalactone | Natural & Kosher Parve |
| gamma Dodecalactone | Natural & Kosher Parve |
| Ginger Oil Chinese | Natural & Kosher |
| Ginger Oil Nigerian English | Natural & Kosher |
| Distilled SAS | |
| Grapefruit Key | Natural WONF & Kosher Parve |
| Hectan-2-One (Nat.) | Natural & Kosher |
| Hexene-3-One-4 | Artificial & Kosher Parve |
| Hexyl Acetate | Natural & Kosher Parve |
| Homo Cyclocitral, beta | Artificial & Kosher Parve |
| Honey Distillate Nat. | Natural & Kosher |
| Ionone Beta | Natural & Kosher Parve |
| Iso Amyl Iso Valerate | Natural & Kosher Parve |
| Iso Butyl Caproate | Natural & Kosher Parve |
| Iso Butyl Furyl Propionate | Artificial & Kosher Parve |
| Iso Fragarone-030 | Artificial & Kosher Parve |
| Iso Fragarone, 1% ETOH ™ | Artificial & Kosher Parve |
| Juniperberry Oil English | Natural & Kosher |
| Distilled SAS | |
| Ketone Mix | Natural & Kosher Parve |
| Kumarone ™ | Artificial & Kosher Parve |
| Lemon Oil 5× Sas | Natural & Kosher |
| Lemon Oil Terpeneless Sas | Natural & Kosher |
| Lemonless Lemon Key | Natural & Kosher Parve |
| Lime Oil Terpeneless | Natural & Kosher |
| Linalool 75/80% Ex Orange (Nat.) | Natural & Kosher |
| Linalyl Acetate (Nat.) | Natural & Kosher |
| Mangone 5% ETOH ™ | Natural & Kosher Parve |
| Methional | Natural & Non-Kosher |
| Methyl Butyric Acid (2) | Natural & Kosher Parve |
| Methyl Ketones (Nat.) | Natural & Kosher |
| Methyl Oxycyclosulfide 719 | Artificial & Kosher Parve |
| Natural Flavor (99% Vanillin) | Natural & Kosher Parve |
| Nat. Cocoa Butter Distillate | Natural |
| Nonan-2-One (Nat.) | Natural & Kosher |
| Octanal 35% (Nat.) | Natural & Kosher |
| Octen-4-one-2 | Artificial & Kosher Parve |
| Olibanum Oil English Distilled SAS | Natural & Kosher |
| Orange Oil 15× Decolorized M3706 | Natural & Kosher |
| Orange Oil 950 (10×) | Natural & Kosher |
| Orange Oil Terpeneless 2501 | Natural & Kosher |
| Oxaromate-884 | Artificial & Kosher Parve |
| Oxycyclothione-030 | Artificial & Kosher Parve |
| Paradiff ™ 0.01% ETOHGR | Natural & Kosher Parve |
| Paradiff ™ 0.01% Grapefruit Oil | Natural & Kosher Parve |
| Peach Flavor Key | Natural & Kosher Parve |
| Peppermint Oil Redistilled Yakima | Natural & Kosher |
| Peppermint Oil Spec. Fractions | Parve |
| Phenyl Ethyl 2-Methyl Butyrate | Natural & Kosher Parve |
| Phenyl Ethyl Acetate | Natural & Kosher Parve |
| Phenyl Ethyl Alcohol | Natural & Kosher Parve |
| Phenyl Oxaromate-681 | Artificial & Kosher Parve |
| Pimento Berry Oil English | Natural & Kosher |
| Distilled SAS | |
| Pimento Leaf Oil | Natural & Kosher |
| Pimento Leaf Oil Cleaned | Natural & Kosher |
| Pineapple Compound 15% ETOH GR | Natural & Kosher Parve |
| Pineapple Compound 15% PG | Natural & Kosher Parve |
| Popcorn Chemical | Artificial & Kosher Parve |
| Propionic Acid | Natural & Kosher Parve |
| Raspberry Flavor Key | Natural & Kosher Parve |
| Robustone 1.0% ETOH ™ | Natural & Kosher Parve |
| Robustone ™ | Artificial & Kosher Parve |
| Schinus Molle Oil | Natural & Kosher |
| Sclareolide | Natural & Kosher parve |
| Sesame Distillate Nat. | Natural & Kosher |
| Sinensals (Nat.) | Natural & Kosher |
| Starter Distillate 15× W/S | Natural & Kosher Dairy |
| Strawberriff | Artificial & Kosher Parve |
| Strawberry Base | Natural & Kosher Parve |
| Strawberry Flavor Key | Natural & Kosher Parve |
| Succinic Acid | Natural & Kosher Parve |
| Sulfurome-015 | Artificial & Kosher Parve |
| Sweetness Modifier | Natural & Kosher Parve |
| Tetrahydro Terrazine-014 ™ | Artificial & Kosher Parve |
| Thionol-935 | Artificial & Kosher parve |
| Thionol-966 | Artificial & Kosher Parve |
| trans-2-Hexenal | Natural & Kosher Parve |
| Trimenal Acetate 399 1% ETOH ™ | Artificial & Kosher Parve |
| Tropical Fruit Key Base | Natural & Kosher Parve |
| Undecan-2-One (Nat.) | Natural & Kosher |
| Varamol-106 10% ETOH | Artificial & Kosher Parve |
| Varamol-106 10% NEBM5 | Artificial & Kosher Parve |
| Varamol-106 10% PG | Artificial & Kosher Parve |

The following examples demonstrate the solutions of the present invention. However, it is to be understood that these examples are for illustrative purposes only and do not purport to be wholly definitive as to conditions and scope.

### EXAMPLES

In the examples below, certain chemical ingredients are identified by the following abbreviations.
HPMC: Hydroxylpropyl MethylCellulose
EDTA: Ethylene-Diamine Tetraacetic Acid
BAK: Benzalkonium Chloride, commercially available from Sigma Corp.
PHMB: PolyHexaMethylene Biguanide
Dequest® 2016: Tetrasodium phosphate, (1-hydoxyethylidene)diphosphonic acid, sodium salt, available from Monsanto Co.
Tetronic® 1107: poloxamine surfactant, a tetrafunctional block copolymer surfactant, commercially available from BASF
Pluronic® P123: poloxamine surfactant, a difunctional block copolymer surfactant, commercially available from BASF
Polymer JR®: cationic polysaccharide, polyquaternium 10
Alexidine 2HCl: quaternary ammonium salts. 1,1′-Hexamethylene-bis[5-(2-ethylhexyl)biguanide]

### Example 1

### Opcon-A® Eye Drops with a Sweetener

An ophthalmic solution of Opcon-A® eye drops with sucralose was prepared with the following formulation shown below in Table 3. Opcon-A® Itching and Redness Reliever Eye Drops combine an antihistamine for itch relief with a redness reliever. Available without a prescription, Opcon-A® eye drops relieve the itching and redness caused by pollen, ragweed, grass, animal hair, and dander. The flavoring agent, sucralose, was added by mixing the indicated amount with prepared Opcon-A® eye drops.

TABLE 3

| Ingredient | % w/w |
|---|---|
| Naphazoline HCl | 0.027 |
| Pheniramine maleate | 0.315 |
| HPMC | 0.500 |
| EDTA | 0.100 |
| BAK | 0.010 |
| Boric acid | |
| Sucralose | 2.000 |

### Example 2

### Flavored Opcon-A® Eye Drop Formulation

An ophthalmic solution for Opcon-A® eye drop formulation with orange flavor was prepared with the following formulation shown below in Table 4. The orange flavoring agent was added by mixing the indicated amount with prepared Opcon-A® eye drops.

TABLE 4

| Ingredient | % w/w |
|---|---|
| Naphazoline HCl | 0.027 |
| Pheniramine maleate | 0.315 |
| HPMC | 0.500 |
| EDTA | 0.100 |
| BAK | 0.010 |
| Citric Acid | 0.300 |
| Sucralose | 2.000 |
| Orange Flavor | 0.400 |

### Example 3

### Pharmaceutical Composition (Brimonidine Tartrate) with a Sweetener

An ophthalmic solution of pharmaceutical composition for glaucoma with sucralose was prepared with the following formulation shown below in Table 5. Brimonidine acts on receptors (alpha-receptors) in the blood vessels of the eye causing them to constrict. These blood vessels control the production of the watery fluid that fills the rear of the eye. When the blood vessels constrict, there is a decrease in the production of this watery fluid. Brimonidine is used in the treatment of glaucoma. This is a condition where the fluid drainage from the eye is impaired, resulting in fluid build-up and increased pressure in the eye. Sucralose was added by mixing the indicated amount with prepared Brimonidine Tartrate.

TABLE 5

| Ingredient | % w/w |
|---|---|
| Brimonidine Tartrate | 0.200 |
| BAK | 0.050 |
| Sucralose | 2.000 |

### Example 4

### Flavored Pharmaceutical Composition (Brimonidine Tartrate)

An ophthalmic solution of pharmaceutical composition for glaucoma with lemon flavor was prepared with the following formulation shown below in Table 6. Lemon flavor was added by mixing the indicated amount with prepared Brimonidine Tartrate.

TABLE 6

| Ingredient | % w/w |
|---|---|
| Brimonidine Tartrate | 0.200 |
| BAK | 0.050 |
| Citric Acid | 0.300 |
| Sucralose | 2.000 |
| Lemon Flavor | 0.400 |

### Example 5

### Multi-purpose Solution for Contact Lenses with a Sweetener

A multi-purpose solution for contact lenses with sucralose was prepared with the following formulation shown below in Table 7. Sucralose was added by mixing the indicated amount with prepared Multi-purpose solution for contact lenses.

TABLE 7

| Ingredient | % w/w |
|---|---|
| Sucralose | 1.00 |
| Tetronic 1107 | 1.00 |
| Sodium Borate | 0.09 |
| Boric Acid | 0.64 |
| EDTA | 0.11 |
| PHMB | 1.0 ppm |
| Dequest 2016 | 0.03 |
| Sodium Chloride | 0.49 |
| Purified Water | Q.S. to 100 gm |

### Example 6

### Flavored Multi-purpose Solution for Contact Lenses

A multi-purpose solution for contact lenses with cocoa flavor was prepared with the following formulation shown below in Table 8. Cocoa flavor was added by mixing the indicated amount with prepared Multi-purpose solution for contact lenses.

TABLE 8

| Ingredient | % w/w |
|---|---|
| Sucralose | 1.00 |
| Tetronic 1107 | 1.00 |
| Sodium Borate | 0.09 |
| Citric Acid | 0.30 |
| Cocoa Flavor | 0.50 |
| EDTA | 0.11 |
| PHMB | 1.0 ppm |
| Dequest 2016 | 0.03 |
| Sodium Chloride | 0.49 |
| Purified Water | Q.S. to 100 gm |

### Example 7

### ReNu MultiPlus® and ReNu No-Rub® with a Sweetener

A multi-purpose solution for contact lenses (ReNu MultiPlus® and ReNu No-Rub®) with sucralose was prepared with following formulation shown in below Table 9. ReNu No-Rub® solution provides sustained comfort yet cleans, disinfects, rinses, stores, and removes protein daily for soft contact lenses without the need to rub. It fights contact lens dryness and delivers sustained comfort. Sucralose was added by mixing the indicated amount with prepared ReNu MultiPlus® and ReNu No-Rub®.

TABLE 9

| | ReNu MultiPlus ® | ReNu No-Rub ® |
|---|---|---|
| Ingredient | (% w/w) | (% w/w) |
| Pluronic P123 | — | 2.00 |
| Tetronic 1107 | 1.00 | 1.00 |
| Sodium Chloride | 0.49 | 0.09 |
| Boric Acid | 0.64 | 0.85 |
| Sodium Borate | 0.09 | — |
| EDTA | 0.11 | — |
| Sodium Phosphate | — | 0.15 |
| (monobasic) | | |
| Sodium Phosphate | — | 0.31 |
| (Dibasic) | | |
| Polymer JR | — | 0.02 |
| PHMB HCl | 1.1 ppm | — |
| Alexidine 2HCl | — | 4.5 ppm |
| Sucralose | 1.00 | 1.00 |
| Dequest 2016 | 0.10 | 0.10 |
| Purified Water | Q.S. to 100 gm | Q.S to 100 gm |

### Example 8

### Flavored ReNu MultiPlus® and ReNu No-Rub® Multi-Purpose Solutions

A multi-purpose solution for contact lenses (ReNu MultiPlus® and ReNu No-Rub® solutions) with watermelon flavor was prepared with following formulation shown in Table 10. Watermelon flavor was added by mixing the indicated amount with prepared ReNu MultiPlus® and ReNu No-Rub® solutions.

TABLE 10

| | ReNu MultiPlus | ReNu No-Rub |
|---|---|---|
| Ingredient | (% w/w) | (% w/w) |
| Pluronic P123 | — | 2.00 |
| Tetronic 1107 | 1.00 | 1.00 |
| Sodium Chloride | 0.49 | 0.09 |
| Boric Acid | 0.64 | 0.85 |
| Sodium Borate | 0.09 | — |
| Boric Acid | 0.64 | 0.85 |
| Sodium Borate | 0.09 | — |
| EDTA | 0.11 | — |
| Sodium Phosphate | — | 0.15 |
| (Monobasic) | | |
| Sodium Phosphate (dibasic) | — | 0.31 |
| Polymer JR | — | 0.02 |
| PHMB HCl | 1.1 ppm | — |
| Alexidine 2HCl | — | 4.5 ppm |
| Sucralose | 1.00 | 1.00 |
| Watermelon Flavor | 0.40 | 0.40 |
| Dequest 2016 | 0.10 | 0.10 |
| Purified Water | Q.S. to 100 gm | Q.S. to 100 gm |

The claimed invention is:

1. A method for indicating dosage of an ophthalmic solution, comprising the step of:
adding a flavoring agent to an ophthalmic solution, wherein the amount of the flavoring agent correlates to the dosage of the ophthalmic solution to be used.

2. The method of claim 1, wherein the flavoring agent is present in an amount to occur taste sensation in a person's mouth when applied to the person's eye.

3. The method of claim 1, wherein the flavoring agent is a sweetener.

4. The method of claim 3, further comprising a flavoring agent selected from the group consisting of a sour flavoring agent, a bitter flavoring agent, and mixtures thereof.

5. The method of claim 3, wherein the flavoring agent is present in an amount from about 0.0001 to 20 weight percent.

6. The method of claim 5, wherein the flavoring agent is present in an amount from about 0.1 to 5 weight percent.

7. The method of claim 3, wherein the sweetener is a natural sugar or a sugar substitute of artificial origin.

8. The method of claim 7, wherein the sweetener is sucrose.

9. The method of claim 7, wherein the sweetener is selected from the group consisting of sucralose, isomalt, aspartame, saccharin, lactitol, or high-fructose corn syrup.

10. The method of claim 9, wherein the sweetener is sucralose.

11. The method of claim 1, wherein the flavoring agent is selected from the group consisting of natural flavors, natural fruit flavors, artificial flavors, artificial fruit flavors, flavor enhancers and mixtures thereof.

12. The method of claim 11, further comprising a flavoring agent selected from the group consisting of a sweetener, a sour flavoring agent, a bitter flavoring agent, and mixtures thereof.

13. The method of claim 11, wherein the flavoring agent is present in an amount from about 0.0001 to 20 weight percent.

14. The method of claim 13, wherein the flavoring agent is present in an amount from about 0.1 to 5 weight percent.

15. The method of claim 1, wherein the ophthalmic solution is selected from a contact lens solution, an eye drop formulation, and a pharmaceutical composition containing at least one active pharmaceutical ingredient for the treatment of an eye disease.

16. A method of administering an ophthalmic solution to a person in need thereof, comprising the step of introducing the ophthalmic solution comprising a flavoring agent to a person's eye until a taste sensation occurs in the person's mouth.

17. The method of claim 16, wherein the flavoring agent is a sweetener.

18. The method of claim 17, further comprising a flavoring agent selected from the group consisting of a sour flavoring agent, a bitter flavoring agent, and mixtures thereof.

19. The method of claim 17, wherein the flavoring agent is present in an amount from about 0.0001 to 20 weight percent.

20. The method of claim 19, wherein the flavoring agent is present in an amount from about 0.1 to 5 weight percent.

21. The method of claim 16, wherein the sweetener is a natural sugar or a sugar substitute of artificial origin.

22. The method of claim 21, wherein the sweetener is sucrose.

23. The method of claim 21, wherein the sweetener is selected from the group consisting of sucralose, isomalt, aspartame, saccharin, lactitol, or high-fructose corn syrup.

24. The method of claim 23, wherein the sweetener is sucralose.

25. The method of claim 16, wherein the flavoring agent is selected from the group consisting of natural flavors, natural fruit flavors, artificial flavors, artificial fruit flavors, flavor enhancers and mixtures thereof.

26. The method of claim 16, wherein the ophthalmic solution is selected from a contact lens solution, an eye drop formulation, and a pharmaceutical composition containing at least one active pharmaceutical ingredient for the treatment of an eye disease.

27. An ophthalmic solution comprising at least one flavoring agent, wherein the amount of the flavoring agent correlates to the dosage of the ophthalmic solution to be used.

28. The ophthalmic solution of claim 27, wherein the flavoring agent is present in an amount to occur taste sensation in a person's mouth when applied to the person's eye.

29. The ophthalmic solution of claim 27, wherein the flavoring agent is a sweetener.

30. The ophthalmic solution of claim 29, further comprising a flavoring agent selected from the group consisting of a sour flavoring agent, a bitter flavoring agent, and mixtures thereof.

31. The ophthalmic solution of claim 30, wherein the flavoring agent is present in an amount from about 0.0001 to 20 weight percent.

32. The ophthalmic solution of claim 31, wherein the flavoring agent is present in an amount from about 0.1 to 5 weight percent.

33. The ophthalmic solution of claim 29, wherein the sweetener is a natural sugar or a sugar substitute of artificial origin.

34. The ophthalmic solution of claim 33, wherein the sweetener is sucrose.

35. The ophthalmic solution of claim 33, wherein the sweetener is selected from the group consisting of sucralose, isomalt, aspartame, saccharin, lactitol, or high-fructose corn syrup.

36. The ophthalmic solution of claim 35, wherein the sweetener is sucralose.

37. The ophthalmic solution of claim 26, wherein the flavoring agent is selected from the group consisting of natural flavors, natural fruit flavors, artificial flavors, artificial fruit flavors, flavor enhancers and mixtures thereof.

38. The ophthalmic solution of claim 26, wherein the ophthalmic solution is selected from a contact lens solution, an eye drop formulation, and a pharmaceutical composition containing at least one active pharmaceutical ingredient for the treatment of an eye disease.
